(19) United States
(12) Patent Application Publication
(10) Pub. No.: US 2017/0143196 A1
(43) Pub. Date: May 25, 2017

(54) SCANNING OPTICAL PROBE

(71) Applicants: Massachusetts Institute of Technology, Cambridge, MA (US); President and Fellows of Harvard College, Cambridge, MA (US)

(72) Inventors: Kaicheng Liang, Cambridge, MA (US); James G. Fujimoto, Medford, MA (US); Hiroshi Mashimo, Lincoln, MA (US); Osman Oguz Ahsen, Cambridge, MA (US); Hsiang-Chieh Lee, Cambridge, MA (US); Michael Gene Giacomelli, Cambridge, MA (US); Zhao Wang, Quincy, MA (US)

(21) Appl. No.: 15/340,530

(22) Filed: Nov. 1, 2016

Related U.S. Application Data
(63) Continuation of application No. PCT/US2015/028844, filed on May 1, 2015.
(60) Provisional application No. 61/987,801, filed on May 2, 2014.

(51) Int. Cl. A61B 1/04; A61B 10/02; A61B 1/012; A61B 5/07; A61B 1/00; A61B 5/00

(52) U.S. Cl. CPC A61B 1/041; A61B 1/00188; A61B 1/00172; A61B 1/00018; A61B 5/0066; A61B 5/0084; A61B 1/012; A61B 1/043; A61B 5/07; A61B 5/742; A61B 10/02

(57) ABSTRACT

The invention pertains to an apparatus and methods of a medical imaging device for obtaining images from the walls of luminal organs or a surgical cavity. The invention is a rigid enclosure that is capable of passage through luminal organs or introduction into surgical cavities, and obtains images by rapidly scanning a focused light beam on the tissue to be imaged and receiving light from the tissue. The invention has at least one beam scanning mechanism and has multiple embodiments of scanning and focusing optics at different regimes of numerical aperture. The invention also describes methods for correcting inaccurate beam scanning. The device is capable of performing imaging, image guided therapy, tissue excision, or other interventional procedures.

### RELATED APPLICATION

This application is a Continuation of International Application No. PCT/US2015/028844, filed May 1, 2015, which designates the U.S., published in English, and claims the benefit of U.S. Provisional Application No. 61/987,801, filed May 2, 2014. The entire teachings of the above applications are incorporated herein by reference.

### GOVERNMENT SUPPORT

This invention was made with government support under Grant No. R01-CA075289 from National Institutes of Health, Grant No. FA9550-10-1-0551 from the Air Force Office of Scientific Research, and Grant No. R01-CA178636 from National Institutes of Health. The government has certain rights in the invention.

### BACKGROUND OF THE INVENTION

Structural and functional changes in the tissue of luminal organs are known to be indicative of disease occurrence and progression. Conventional white light imaging with endoscopes or related devices deliver images of the luminal surface; however, the gross appearance of the luminal wall does not always detect tissue changes below the surface or microscopic changes that may be pathological.

The application of these advanced imaging methods can be limited by the mechanical requirements the delivery and collection of beams of light using an optical fiber in combination with an imaging device that is introduced into a luminal organ or surgical cavity. These optical imaging methods typically require the mechanical scanning of an optical beam over a desired area of the tissue for the intended interrogation. The reconstruction of an image requires that the optical beam be rapidly scanned in an accurate and known fashion. High resolution images further require that the cross section (spot diameter) of the optical beam be relatively small, which compounds the necessity for accurate beam scanning.

### SUMMARY OF THE INVENTION

In an example embodiment, the invention is an apparatus for optical imaging of a luminal organ or surgical cavity. The apparatus comprises a proximal end, including at least one optical connection, and at least one of mechanical connection and an electrical connection, and a distal end that comprises a rigid enclosure. The rigid enclosure includes at least one transparent portion. The distal end includes at least one optical connection, and at least one of mechanical connection and an electrical connection. The apparatus further includes a flexible or semi-flexible tether. The tether includes at least one optical fiber that connects the distal end of the apparatus to the proximal end of the apparatus. The tether further connects at least one of a mechanical connection and an electrical connection of the proximal end to the at least one of mechanical connection and an electrical connection of the distal end. The rigid enclosure comprises at least one focusing optical element in optical communication with the at least one optical fiber. The focusing optical element is configured to direct and focus light from the optical fiber through the transparent portion of the rigid enclosure. The apparatus further includes a scanning mechanism. The scanning mechanism including a rotary actuator configured to perform beam scanning in a rotary direction. The scanning mechanism is further configured to perform beam scanning in a longitudinal direction, wherein the rotary direction and the longitudinal direction are non-parallel.

In another example embodiment, the invention is an apparatus for optical imaging of a luminal organ or surgical cavity. The apparatus comprises a proximal end, including at least one optical connection, and at least one of mechanical connection and an electrical connection. The apparatus further includes a distal end that comprises a rigid enclosure. The rigid enclosure includes at least one transparent portion. The distal end further includes at least one optical connection, and at least one of mechanical connection and an electrical connection. The apparatus further comprises a flexible or semi-flexible tether. The tether includes at least one optical fiber that connects the distal end of the apparatus to the proximal end of the apparatus, the tether further connecting at least one of a mechanical connection and an electrical connection of the proximal end to the at least one of mechanical connection and an electrical connection of the distal end. The rigid enclosure further comprises at least one focusing optical element in optical communication with the at least one optical fiber. The focusing optical element is configured to direct and focus light from the optical fiber through the transparent portion of the rigid enclosure. The apparatus further includes a scanning mechanism. The scanning mechanism includes a rotary actuator, the rotary actuator being configured to perform beam scanning in a rotary direction. In an example embodiment of the apparatus of the present invention, the rigid enclosure includes at least one static landmark configured to detect and correct non-uniform or inaccurate beam scanning. In various example embodiments, the optical fiber remains stationary in the rotary direction with respect to the rigid enclosure during beam scanning.

In another example embodiment, the present invention is a method of optical imaging of a luminal organ or a surgical cavity. The method employs the devices described herein. The method comprises acquiring an optical image of the luminal organ or surgical cavity using any of the devices described herein.

In another example embodiment, the present invention is a method for correcting an optical image of a luminal organ or a surgical cavity. The method employs any of the devices described herein. The method comprises causing any of the devices described herein to scan the luminal organ or surgical cavity and to acquire the optical image of the luminal organ or surgical cavity; detecting a scanning beam position; measuring inaccuracies in the scanning beam position; and, based on the measured inaccuracies in the scanning beam position, controlling the at least one scanning mechanism to correct the optical image.

The scanning mechanisms and actuators employed in the embodiments of the present invention described herein perform precise scanning of the imaging beam in two non-parallel directions. Residual non-uniformity or imprecision of the scanning can be corrected. Furthermore, the integration of interventional procedures, such as tissue biopsy, mucosal resection, therapy, or other procedures within the imaging device can be advantageous in reducing the health care cost and labor associated with performing consecutive procedures, as well as improving the diagnostic correlation or accuracy of the imaging method. The combination of one or more of these factors offer suitable solutions to the shortcomings of the medical imaging devices used in the prior art.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a schematic diagram of an exemplary embodiment of an imaging system of the invention.

FIG. 2A is an illustration of an embodiment of the present invention showing the invention as a rigid enclosure tethered to a semi-flexible catheter that permits gentle bending but retains some rigidity.

FIG. 2B is an illustration of an embodiment of the present invention showing the rigid enclosure tethered to a small diameter and highly flexible catheter that permits multiple bends and has very low rigidity.

FIG. 3A is an illustration of an embodiment of the present invention showing the rigid enclosure containing two beam scanning mechanisms, a rotary actuator and linear actuator, both of which are contained within the enclosure and imparting motion to the focusing optics.

FIG. 3B is an illustration of an embodiment of the present invention showing the rigid enclosure containing a rotary actuator that is connected via a mechanical transduction to a linear actuation inside the enclosure.

FIG. 4A is an illustration of an embodiment of the present invention showing a rotary actuator located at the proximal end of the device, connected to a torque cable and a rotary transduction mechanism in the enclosure to change the rotary speed of the focusing optics relative to the torque cable.

FIG. 4B is an illustration of an embodiment of the present invention showing a rotary actuator and linear actuator located at the proximal end of the device connected to a torque cable which is connected to a rotary transduction mechanism inside the enclosure to change the rotary speed of the focusing optics relative to the torque cable.

FIG. 5A is an illustration of an embodiment of the present invention showing a rotary actuator located at the proximal end of the device, connected to a rotary transduction mechanism in the enclosure to change the rotary speed of the focusing optics, and a mechanical transduction to linear actuation inside the enclosure.

FIG. 5B is an illustration of an embodiment of the present invention showing a rotary actuator located at the proximal end of the device, connected to a rotary transduction mechanism in the enclosure to change the rotary speed of the focusing optics, and a linear actuator located inside the enclosure.

FIG. 6A is an illustration of an embodiment of the present invention showing a linear actuator located at the proximal end of the device and connected to a mechanical transduction to linear actuation inside the enclosure, and a rotary actuator inside the enclosure.

FIG. 6B is an illustration of an embodiment of the present invention showing a rotary actuator located at the proximal end of the device and connected to a mechanical transduction to linear actuation inside the enclosure, and a rotary actuator inside the enclosure.

FIG. 7 is an illustration of an embodiment of the present invention showing a linear actuator located at the proximal end of the device, and a rotary actuator inside the enclosure.

FIG. 8A is a schematic diagram of an embodiment of the present invention that employs a low NA optical design that has the optical focusing components entirely on the central longitudinal axis of the enclosure.

FIG. 8B is a schematic diagram of an embodiment of the present invention that employs a high NA optical design that has optical focusing components held on the central longitudinal axis of the enclosure and also on a radial axis orthogonal to the central longitudinal axis.

FIG. 8C is a schematic diagram of an embodiment of the present invention that employs a high NA optical design that has optical focusing components held on the central longitudinal axis of the enclosure and also on a radial axis orthogonal to the central longitudinal axis and is employing another optical element to split the incoming beam into two directions.

FIG. 9 is an illustration of an embodiment of the present invention showing the rotary actuator proximally located relative to the focusing optics within the enclosure, with the optical beam incoming to the focusing optics from the distal end of the enclosure, due to reflection by one or more reflectors.

FIG. 10A is a schematic diagram of an embodiment of the invention, wherein the longitudinally actuating carriage that is made of a low friction material such as Teflon and has multiple chamfers for reduced friction.

FIG. 10B is a schematic diagram of an embodiment of the invention, wherein the longitudinally actuating carriage that is mounted with low friction ball bearings.

FIG. 11A is an illustration of an embodiment of the present invention wherein the transparent enclosure includes static landmarks for registration that include a 1-dimensional pattern of multiple horizontal fiducial lines.

FIG. 11B is an illustration of an embodiment of the present invention wherein the transparent enclosure includes static landmarks for registration that includes a 2-dimensional pattern.

FIG. 12A is a schematic diagram of an embodiment of the invention illustrating thickness variation in the enclosure along the rotary direction that is presented in a cross-sectional plane.

FIG. 12B is a schematic diagram of an embodiment of the invention illustrating thickness variation in the longitudinal direction that is presented in a plane orthogonal to that in FIG. 12A.

FIG. 13A is a schematic diagram of an embodiment of the present invention that employs stabilization of the enclosure via vacuum ports.

FIG. 13B is a schematic diagram of an embodiment of the present invention that employs excision functionality in the device, in which tissue is pulled into the enclosure by vacuum and excised by a moving cutter.

FIG. 13C is a schematic diagram of an embodiment of the present invention that employs excision functionality in the device, in which tissue is pulled into the enclosure by vacuum and excised by an electrocauterizing or other wire loop.

FIG. 14 is a photograph of one exemplary embodiment of the invention.

FIG. 15 is a photograph of the embodiment in FIG. 14 showing a pneumatic actuator exsufflated.

FIG. 16 is a photograph of the embodiment in FIG. 14 showing the rotary actuator rotating the focusing optics.

### DETAILED DESCRIPTION OF THE INVENTION

A description of example embodiments of the invention follows.

The present invention is an imaging device that can have a rigid transparent enclosure useful for optical imaging of a luminal organ or surgical cavity using optical coherence tomography (OCT), polarization sensitive OCT, Doppler OCT, OCT angiography, scanning laser, two-photon, harmonic, multi-photon, fluorescence, fluorescence lifetime imaging or other methods. In example embodiments, the enclosure is cylindrical and sufficiently small in diameter and longitudinal or axial length to enable introduction into the luminal organ or surgical cavity. The enclosure can be connected to the imaging engine by a tether, which contains at least one optical fiber, and may contain at least one electrical wire, and may contain at least one tube for pneumatic or hydraulic infusion, and may contain at least one mechanical cable such as a torque cable. In various embodiments, an optical beam can be used to scan in at least two non-parallel directions with different fast and slow scanning axes. The actuators that perform the scanning can be located either in the distal portion of the device contained within the enclosure, or they can be outside the device connected to the proximal portion of the device. In various embodiments, the focusing optics can permit either low numerical aperture (NA) or high numerical aperture focusing. Moreover, in various embodiments the device can include features that enable for image guided interventional procedures, such as tissue biopsy, mucosal resection, therapy, or other procedures.

In other embodiments, the invention is a method for correcting non-uniform or inaccurate beam scanning to obtain undistorted images. In some embodiments of the invention, some of the actuators can have sensors that measure the inaccuracies in the intended scanning. In other embodiments the enclosure can have fiducial markers that can be used to measure the inaccuracies in the intended scanning. In other embodiments the enclosure can have physical variations in its thickness or other properties, which can be detected by the imaging system during the scanning and can be used to measure the inaccuracies in the intended scanning. In all of these embodiments, the measured inaccuracy in the scanning can be used to control the scanning and or correct for the distortions in the images due to non-uniform or inaccurate beam scanning by post processing image data. In further embodiments, the present invention includes devices and methods for stabilizing or detecting and subsequently correcting relative motion between the enclosure and the surrounding luminal tissue.

The beam scanning should be conducted along at least two non-parallel axes in order to be able to generate en face view of volumetric images, which are two or three dimensional data sets, of tissue regions. At least one of the scanning directions or axes is preferred to be aligned with the axis of the imaging device and luminal organ that often can be considered as having a cylindrical shape. This direction will be termed the axial or longitudinal direction and the scanning in this direction termed the axial or longitudinal scan. A second scanning direction is substantially, locally orthogonal to the first direction in order to maximize the efficiency of the two dimensional scanning. This second scan direction is preferred to be along the circumference of the cylinder. This second direction will be termed the rotary or angular direction and the scanning in this direction termed the rotary or angular scan. Furthermore, in at least one of the directions it is preferred that the scan be conducted in a very rapid manner compared to other direction, in order to minimize motion related artifacts in the images related to this particular direction.

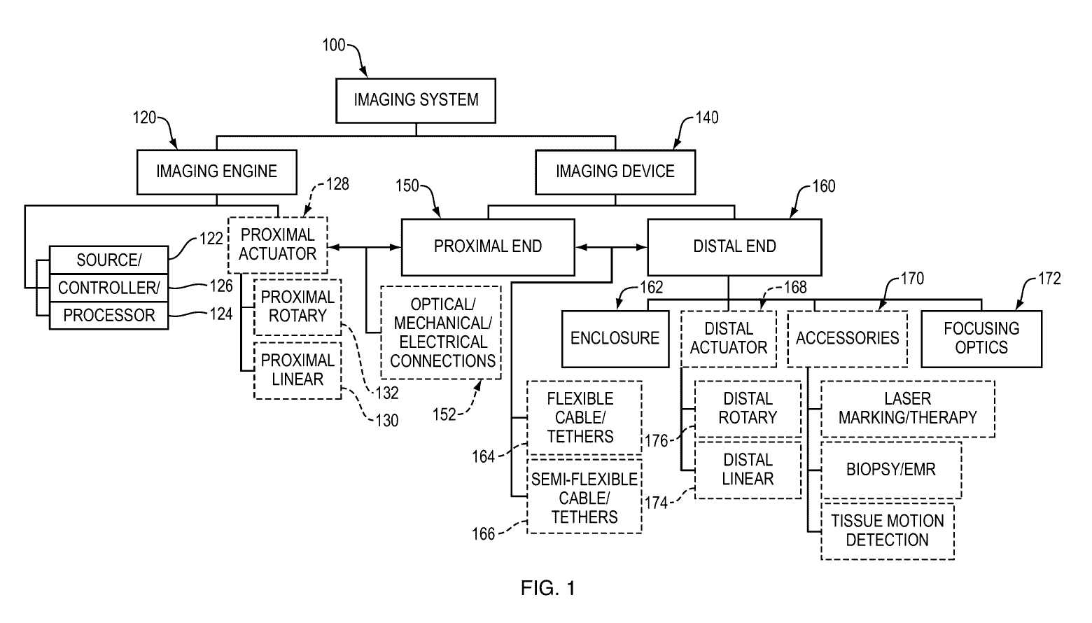

FIG. 1 is a schematic illustration and components of an exemplary imaging system 100 that can employ the imaging device described in this invention. The boxes outlined with dashed lines represent optional components.

Referring to FIG. 1, the imaging system 100 comprises an imaging device 140 and an imaging engine 120. The imaging engine 120 can include one or more light sources 122, one or more processor units 124, one or more controllers 126, and may further include one or more actuators 128. The processing unit is used for signal acquisition, processing, display, storage and other computerized tasks, and can be a computer. The controller unit can also be a computer, and can be used for controlling the light source, actuators or other components, and can be used to achieve synchronization of the movement between different actuators employed in the system.

The imaging system 100 can include an optional proximal actuator 130, employed within the imaging engine 120 of some of the embodiments. The actuator 128 can be a linear actuator 130 or rotary actuator 132 or a combination of both.

The imaging device 140 includes a proximal end 150 and a distal end 160. The proximal end 150 refers to the portion of the imaging device 140 that is closest to and readily accessible by the operator. The proximal end 150 can, for example, be disposed outside the luminal organ and surgical cavity. It is connected to the imaging engine using a tether with at least one optical fiber and may include mechanical and electrical connections 152. The distal end 160 of the imaging device 140 refers to a rigid enclosure 162 and the components that are contained within enclosure 162. The proximal and distal ends 150 and 160, respectively, of the imaging device 140 are connected with a flexible or semi-flexible tethers or cables 164 and 166. The distal end 160 of the imaging device 140 can include a housing (not indicated in FIG. 1), one or more distal actuators 168, accessory components 170, and an optical system, for example focusing optics 172. A distal actuator is employed in the distal end of some of the embodiments, and can be a linear actuator 174, a rotary actuator 176, or a combination of both. The accessory components 170 can increase the functionality of the imaging device or increase the accuracy of the beam scanning.

In the following, more detailed descriptions of these components and various embodiments that employ one or more of these components will be given.

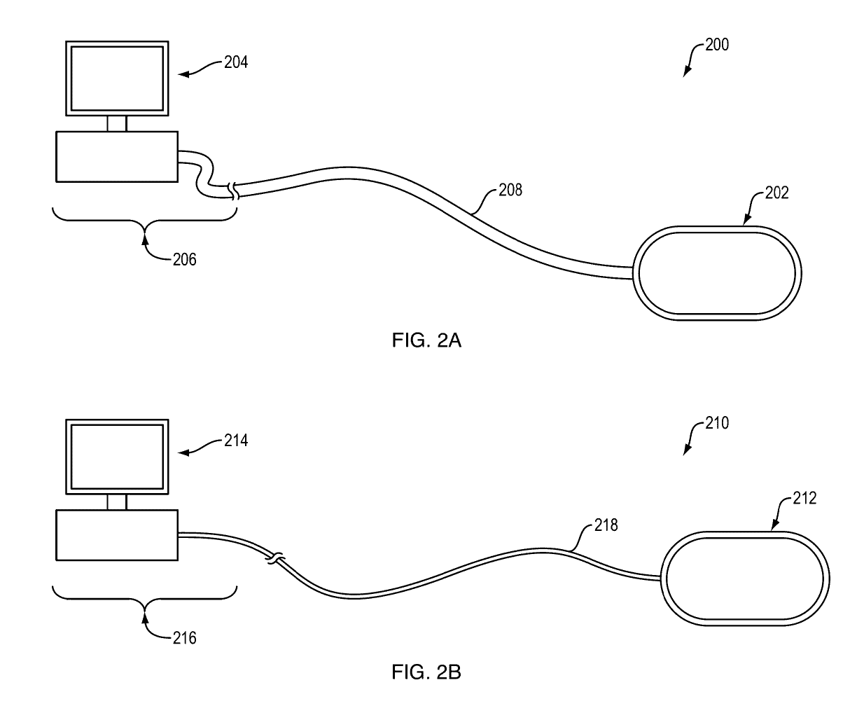

FIG. 2A and FIG. 2B are schematic illustrations showing two exemplary embodiments of tethers that can be employed in the devices described herein. The tether connects the imaging device enclosure to the imaging instrument or a patient interface module of the imaging instrument. In FIG. 2A, the device 200 includes the enclosure 202, connected to a imaging instrument or a patient interface module 204 at the proximal end 206 of the device 200 by a tether 208 that retains sufficient rigidity such that the catheter tolerates only gentle bending. This embodiment permits robust placement of the device in a luminal organ when manipulated from the proximal end of the tether, during a procedure such as endoscopy or surgery when the patient is under sedation or anesthesia. In FIG. 2B, the device 210 includes the enclosure 212, connected to the imaging instrument or a patient interface module 214 at the proximal end 216 of the device 210 by a tether 218 that is highly flexible and soft, and able to retain multiple contortions with little mechanical resistance. This embodiment can be passed through a luminal organ such as the esophagus and other luminal organs without inducing discomfort to the patient who can be conscious and unsedated. For example, the embodiment of the device 210 shown in FIG. 2B can be swallowed when used for upper gastrointestinal (GI) imaging. The device can be positioned in, or retrieved from, the esophagus using the tether 218.

The tethers 208 and 218 contain at least one optical fiber used to deliver or receive light. For OCT imaging, the optical fiber conducts light from the laser source to the distal end of the device and collects back reflected or backscattered light from the tissue being imaged. For multiphoton imaging, the fiber may be a low dispersion hollow core or photonic crystal fiber. For multiphoton imaging, the fiber may be a dual core/clad fiber where the inner core is used to conduct short pulse excitation light from the instrument to the distal end of the device and the outer core/cladding is used to collect fluorescence or second harmonic generation or other forms of nonlinear-generated light from the tissue for detection. The tethers further connect at least one of a mechanical connection and an electrical connection of the proximal end to at least one of a mechanical connection and an electrical connection of the distal end of the devices 200 and 210 (not shown).

In the embodiments shown in FIGS. 2A and 2B, the enclosures 202 and 212 can be cylindrically shaped, having a transparent window section (not shown).

In various embodiments, devices described herein can scan an optical beam in at least two non-parallel directions with different preferred fast and slow scanning axes. The scanning actuation mechanisms can be located either in the proximal or in the distal end of the imaging device. Distal actuation may be more stable than proximal actuation, because distal actuation is applied directly on the object to be actuated, while proximal actuation is applied via a long, connecting mechanical cable such as a torque cable, which is subject to stretch, twist and other deformations that result in mechanical instability of the actuation. Proximal actuation also may have limits in speed because of mechanical instabilities when torque cables are actuated at high speed. Two scanning mechanisms may be employed, in various embodiments, such as a fast rotary or angular scan and a slow longitudinal scan.

For optical imaging applications, in order to acquire image data without loss of resolution set by the optical spot size, the separation between individual samples/pixels/OCT A-scans along a particular direction is determined by the Nyquist criteria. For the fast-rotary scan, then number of samples N_{Nyquist} in one rotation can be calculated as:

$${N}_{nyquist} = 2 \times \frac{C}{ω}$$

Where C represents the circumference of the cylinder or rotary scan and ω represents the spot size/transverse resolution of the selected optical imaging system. For this condition to hold, the ratio of data sampling rate S (or A-scan rate in OCT) over frequency of rotary scanning f should be set in order to obtain a sufficient number of measurements during each rotary scan. This yields the condition:

$${N}_{nyquist} = 2 \times \frac{C}{ω} = \frac{S}{f} .$$

This condition also determines a fast rotary scanning frequency, given the data sampling rate, cylinder circumference and spot size:

$$f = \frac{S}{2} \frac{ω}{C}$$

Alternately, this can be expressed as a velocity V_{C} of the scanning optical beam along the circumference of the cylinder:

$${V}_{C} = C \cdot f = \frac{S ω}{2}$$

Similarly for the slow scan direction, which is the axial or longitudinal direction, the Nyquist criteria yields:

$${V}_{L} = f \times \frac{ω}{2}$$

where V_{L} is the translation velocity or scan speed in the axial or longitudinal direction.

For OCT imaging an example A-scan rate would be S=1,000,000 (1 MHz) for state of the art OCT instruments. For a rotary scan with C=30 mm and spot size of ω=20 um, one requires a rotary scan frequency of 330 Hz, corresponding to a scanning speed V_{C}=9,900 mm/s along the circumference. Conversely the axial or longitudinal translation speed is V_{L}=3.3 mm/sec to satisfy the Nyquist criteria. Although this example is presented assuming an A-scan rate of 1 MHz, it is noted the OCT imaging can be performed at different rates which can be 50 kHz to a few MHz. It is also noted that the circumference and spot size may be different depending on the application.

This example illustrates parameters for the fast scan direction and the slow scan directions. The invention enables accurate beam scanning where the scan velocities in the fast and slow scan directions are different by orders of magnitude.

It should be mentioned that although the Nyquist criteria provides general guidelines for imaging, variations from the above mentioned examples are possible depending on the resolution and image acquisition time requirements of the application. For example, for certain imaging modalities such as Doppler OCT or OCT angiography, one requires oversampling compared to the Nyquist criteria with time separation between samples. For the OCT example given previously assuming the same sampling, spot size and circumference, OCT angiography would require an oversampling factor of ˜5× in the longitudinal direction, between sequential rotary scans, which requires a lower longitudinal translation speed of 0.3 mm/sec.

In addition, for all of the aforementioned imaging modalities denser/sparser samples compared to Nyquist criteria may be acquired in order to perform averaging to increase image quality and/or to perform an initial preview or scout scan to identify regions of interest for further interrogation and/or for calibrating/determining acquisition parameters or settings, and/or confirming the positioning of the imaging device at a desired location.

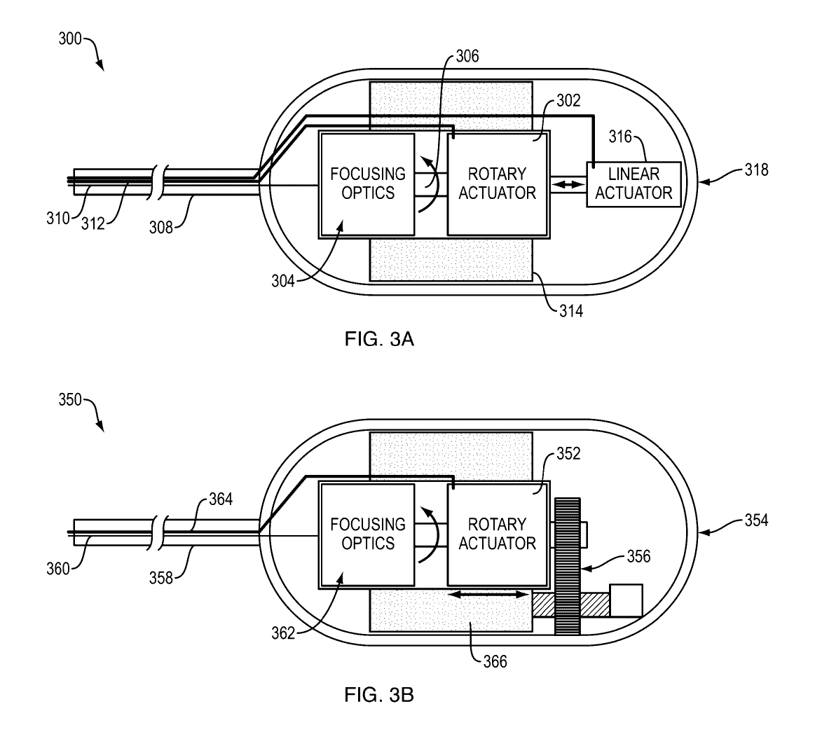

FIG. 3A through FIG. 9 are schematic illustrations showing the internal layout of the exemplary embodiments of the imaging device of the present invention. In the example embodiments, the focusing optics is actuated by at least one of a rotary actuator and a linear actuator. The rotary actuator enables fast beam scanning and the linear actuator enables slow beam scanning in the rotary and longitudinal directions respectively. A rotary or linear actuator may be specified to be located at either the proximal end of the device, or at the distal end of the device inside the enclosure. A proximal rotary actuator may be an electromagnetic actuator or other actuator. A distal rotary actuator may be an electromagnetic or piezoelectric or pneumatic or hydraulic or other actuator. A proximal linear actuator may be an electromagnetic actuator or other actuator. A distal linear actuator may be an electromagnetic or piezoelectric or pneumatic or hydraulic or thermal or shape memory alloy or other actuator. In all of the following embodiments, the rotary actuator rotates the focusing optics about the central longitudinal axis of the cylindrical enclosure and projects an optical beam at a substantially right angle to the longitudinal axis, towards and focusing through the transparent cylindrical wall of the enclosure. In some applications, it is desirable that the optical beam be directed a few degrees from perpendicular to the transparent cylindrical wall in order to avoid parasitic optical reflections from the cylindrical wall. The angle deviation from perpendicular can be calculated based on the required minimum back reflection and the numerical aperture or spot size of the optical beam. The tether contains at least one optical fiber. The tether may also contain at least one of a torque cable, electrical cable or a pneumatic/hydraulic insufflation tube or other control cable for initiating actuation to the rotary actuator and linear actuator, which have an electromagnetic or piezoelectric or pneumatic or hydraulic or other mechanism. If a linear actuator is present in the embodiment, the linear actuator imparts a longitudinal translation to the rotary actuator and focusing optics, which are mounted on a moving carriage having low frictional contact with the cylindrical enclosure wall. Low frictional contact may be achieved by a low friction interface, such as a Teflon contact surface or other low friction material combination, sliding bearings, rolling bearings, or other known method. If the linear actuator is pneumatically actuated, the actuator may be directly inflated and deflated by an insufflation tube which is contained in the tether. Alternately, the pneumatic actuator may be sealed entirely and expanded/compressed without necessity for a separate insufflation tube by modifying the pressure in the enclosure, which can be achieved by exerting a vacuum or pressure via the catheter sheath. If the linear actuator is hydraulically actuated, the actuator may first be completely exsufflated of air by a syringe pump or other exsufflation device, and then filled with incompressible fluid by means of a 3-way luer stopcock or other stopcock device.

In all of example embodiments shown in FIGS. 3A through 9, proximal linear actuation may also be performed directly by mechanical pulling or pushing of the tether by the operator without employing a separate linear actuator.

Proximal rotary actuation on a torque cable can be coupled to distal rotary actuation by means of a gear transduction or other rotary transduction which increases rotary frequency. Typically there is a limit on proximal rotation actuation speed of about 100-200 Hz due to mechanical constraints on proximal actuation of a long torque cable which becomes unstable and has excessive friction at high rotary speeds. For high speed high pixel resolution imaging systems it can be necessary to perform rotary scanning at speeds faster than these values. To overcome this limitation, the rotary transduction mechanism employed in some of the embodiments produces a multiplier effect on the actuated speed and delivers higher rotary speeds for rotary scanning. In one embodiment, the torque cable for the rotary actuator can be connected to the primary gear with a diameter of Rₚ located at the distal end. This primary gear then can be connected to the secondary gear with a diameter Rₛ, which can be employed as the distal rotary actuation mechanism to actuate the carriage. In this configuration the ratio of distal rotary actuation frequency to proximal rotary actuation frequency will be equal to Rₚ/Rₛ. In these embodiments, Rₚ can be chosen larger than Rₛ, which increases the distal rotation scan frequency compared to the proximal rotary scan and torque cable rotation frequency. In some other embodiments it can be necessary to perform rotary scanning at speeds slower than the actuated speed; in these embodiments, Rₚ can be chosen smaller than Rₛ. It is recognized that more than two gears, systems such as planetary gears and other methods for transforming rotary speed can also be used. In a related embodiment the rotary actuation frequency achievable by the proximal rotary actuation might be deemed to be sufficient for the application so that the rotary transduction mechanism is not needed. In this embodiment the rotary actuator can directly transmit the rotary actuation to the distal end of the device with a torque cable.

In certain embodiments, a distal rotary actuator is employed to realize rapid distal rotary scan, and a distal linear actuator is employed to realize slow longitudinal scan. Referring to FIG. 3A, the device 300 includes a rotary actuator configured to rotate focusing optics 304 that is mounted on the actuator shaft 306. The tether 308 contains at least one optical fiber 310 having an end aligned to the focusing optics 304, and at least one control cable 312 for initiating actuation to the rotary actuator 302. The rotary actuator 302 and focusing optics 304 are translated in unison on a carriage 314 by a linear actuator 316. The carriage 314 slides or rolls (not shown) on a very low friction contact with the wall of the enclosure 318. By translating the carriage 314, the linear actuator 316 scans a beam produced by the focusing optics 304 along the longitudinal axis of the enclosure 318.

In certain embodiments, a single distal rotary actuator is employed to realize both rapid distal rotary scan and distal slow longitudinal scan. In these embodiments the rotary scan is realized with a rotary actuator located in the device enclosure and the longitudinal scan is realized with a linear actuation that is produced by a mechanically coupling or other transduction from the rotary actuator. In one embodiment, axial actuation can be coupled to the rotation by means of a gear reduction or other rotary transduction mechanism that is connected to a lead screw and carriage or other mechanism for transducing rotary to linear motion. Referring to FIG. 3B, the device 350 includes the rotary actuator 352 located inside the enclosure 354 and coupled to a mechanical transduction mechanism 356 that transduces the rapid rotary actuation to slow linear actuation. The tether 358 contains at least one optical fiber 360 having an end aligned to the focusing optics 362, and at least one torque cable, electrical cable or a pneumatic/hydraulic insufflation tube or other control cable 364 for initiating actuation to the rotary actuator 352. The rotary actuator 352 and focusing optics 362 are translated in unison on a low-friction carriage 366 by the transduced linear actuation.

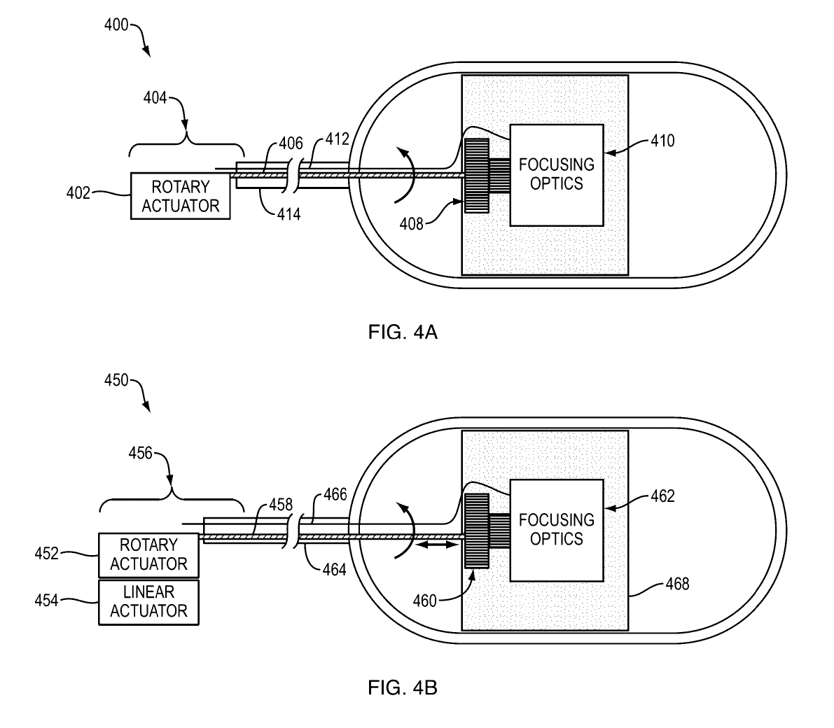

In other embodiments, a single proximal rotary actuator is employed to realize rapid proximal rotary scan. In these embodiments the distal rotary scan is realized with a rotary actuator at the proximal end of the device which transmits rotary actuation to the distal end of the device with a torque cable. The torque cable is connected to a gear assembly at the distal end which increases the rotary speed of the actuation. Rotary speeds for long torque cables are limited and this embodiment enables high rotary speeds required for fast rotary scanning. Referring to FIG. 4A, the device 400 includes the rotary actuator 402 located at the proximal end 404 of the device 400. The rotary actuator 402 rotates a mechanical cable 406 such as a torque cable that is connected to a rotary transducer 408 that multiplies the rotation speed to the focusing optics 410. The tether 414 (for example, a catheter sheath) contains at least one optical fiber 412 having an end aligned to the focusing optics 410, and at least one mechanical cable 406. This embodiment can include a single scanning mechanism, namely rotary scanning mechanism. In a related embodiment, axial translation of the focusing optics is realized by mechanical pulling or pushing of the torque cable by the operator. In another related embodiment, axial translation of the focusing optics is realized by mechanical pulling or pushing of the tether by the operator, which translates the entire enclosure.

In other embodiments, a proximal rotary actuator is employed to realize rapid proximal rotary scan, and a proximal linear actuator is employed to realize proximal slow longitudinal scan. Referring to FIG. 4B, the device 450 includes the rotary actuator 452 and linear actuator 454 is located at the proximal end 456 of the device 450. Both rotary and linear actuators 452 and 454 actuate a mechanical cable 458, such as a torque cable, that is connected to a rotary transducer mechanism 460 that multiplies the rotation speed to the focusing optics 462. The tether 464 (for example, a catheter sheath) contains at least one optical fiber 466 having an end aligned to the focusing optics 462, and at least one mechanical cable 458. The focusing optics 462 is translated on a low-friction carriage 468 by the linear actuator.

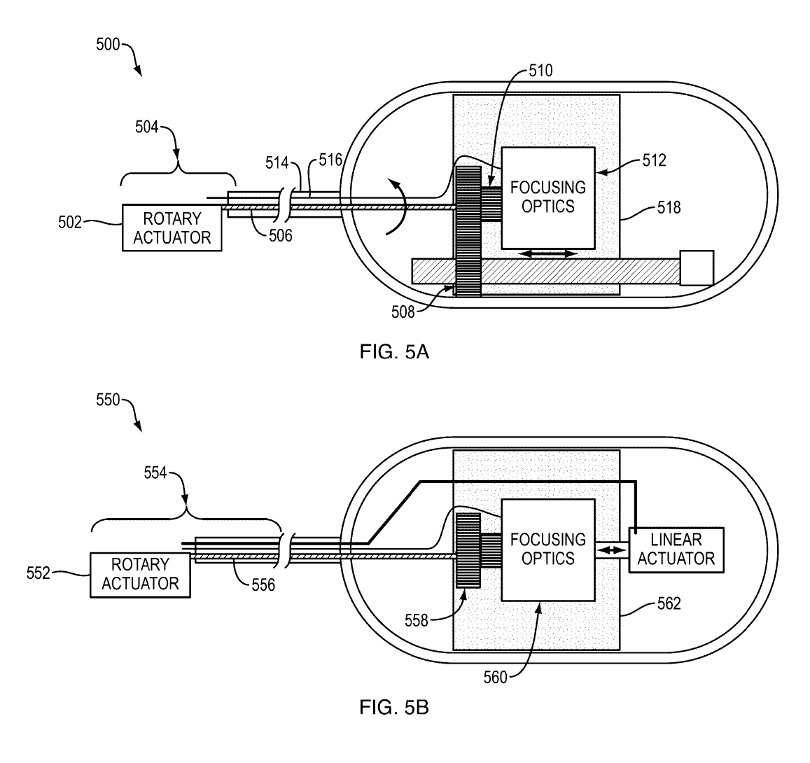

In other embodiments, a single proximal rotary actuator is employed to realize rapid proximal rotary scan and slow distal longitudinal scan. Referring to FIG. 5A, the device 500 includes the rotary actuator 502 is located at the proximal end 504 of the device 500. The rotary actuator 502 actuates a mechanical cable 506, such as a torque cable, that is connected simultaneously to a mechanical transducer mechanism 508 that transduces the rapid rotary actuation to slow linear actuation, and to a gear increasing mechanism 510 that multiplies the rotation speed to the focusing optics 512. The tether 514, for example a catheter sheath, contains at least one optical fiber 516 having an end aligned to the focusing optics 512, and at least one mechanical cable 506. The focusing optics 512 is translated on a low-friction carriage 518 by the linear actuation.

In other embodiments, a proximal rotary actuator is employed to realize rapid proximal rotary scan and a distal linear actuator is employed to realize slow distal longitudinal scan. Referring to FIG. 5B, the device 550 include the rotary actuator 552 located at the proximal end 554 of the device 550. The rotary actuator 552 actuates a mechanical cable 556, such as a torque cable, that is connected to a gear increasing mechanism 558 that multiplies the rotation speed to the focusing optics 560. The focusing optics 560 can be translated on a low-friction carriage 562 by the linear actuator 562.

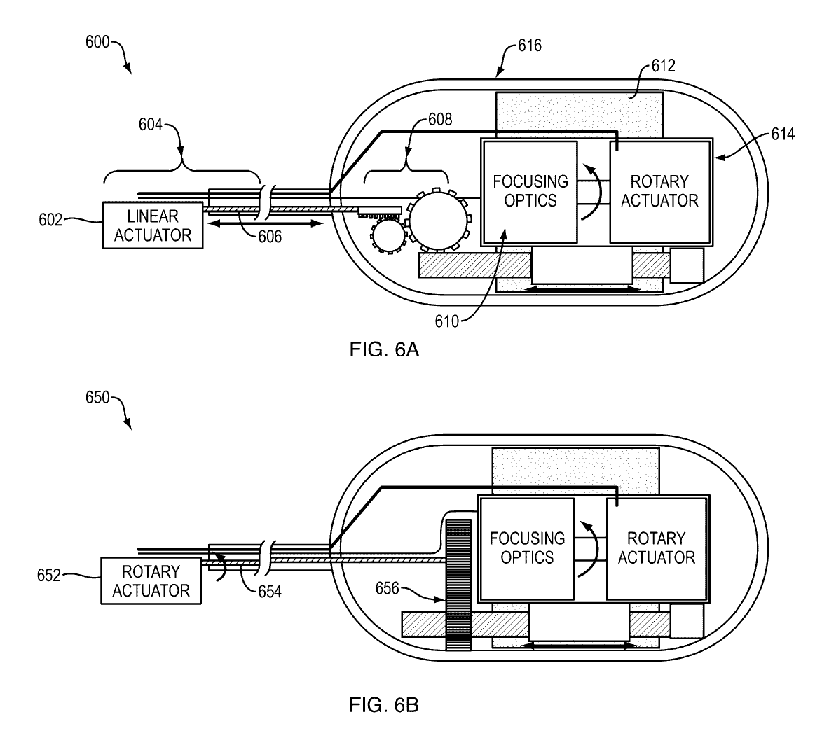

In other embodiments, a distal rotary actuator is employed to realize rapid distal rotary scan and a proximal linear actuator is employed to realize slow proximal longitudinal scan. Referring to FIG. 6A, the device 600 includes the linear actuator 602 located at the proximal end 604 of the device 600. The linear actuator 602 actuates a mechanical cable 606, such as a torque cable, that is connected to a mechanical transducer mechanism 608 that transduces the rapid linear actuation to a slow linear actuation. The focusing optics 610 is translated on a low-friction carriage 612. A rotary actuator 614 is located inside the enclosure 616 and rotates the focusing optics 610.

In other embodiments, a distal rotary actuator is employed to realize rapid distal rotary scan and a proximal rotary actuator is employed to realize slow distal longitudinal scan. Referring to FIG. 6B, the device 650 is an embodiment similar to the device 600 shown in FIG. 6A. However, in the device 650, a proximal rotary actuator 652 (instead of a proximal linear actuator) actuates a mechanical cable 654 (for example, a torque cable) that is connected to a mechanical transducer mechanism 656 that transduces the rapid rotary actuation to a slow linear actuation.

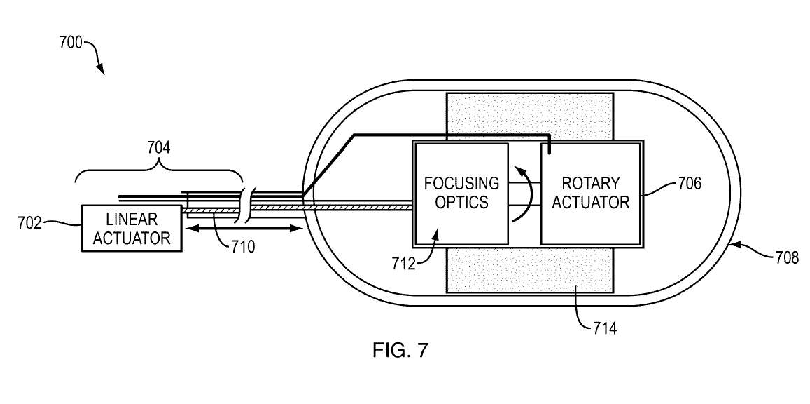

In other embodiments, a distal rotary actuator is employed to realize rapid distal rotary scan and a proximal linear actuator is employed to realize slow proximal longitudinal scan with no mechanical transduction. Referring to FIG. 7, the device 700 includes the linear actuator 702 located at the proximal end 704 of the device 700. The rotary actuator 706 is located inside the enclosure 708. The linear actuator 702 actuates a mechanical cable 710, such as a torque cable, that is connected to the focusing optics 712. The focusing optics 712 is translated on a low-friction carriage 714 that also contains the rotary actuator 706. The focusing optics 712 is rotated by the rotary actuator 706.

In any of the above-described embodiments, the optical design may be either low numerical aperture (NA) for larger beam spot size or high NA for smaller beam spot size. In the low NA embodiment, the focusing optics are entirely along the central longitudinal axis that is being rotated by the rotary actuator, and a mirror bends the beam at a right angle after the focusing optics, such that the working distance of the optical focus is approximately half the diameter of the enclosure. In the high NA embodiment, the rotary actuator rotates a mirror that bends a non-focusing beam at a right angle into the focusing optics, which are held at a radial axis orthogonal to the longitudinal axis, such that the working distance of the optical focus is significantly smaller than the radius of the enclosure. The optical design may include a collimating beam prior to the focusing optics, such that the longitudinal translation of the rotary actuator does not change the optical focus and only leads to a change in optical path length. For all of the latter methods, the high NA embodiment is important for sufficient collection of electromagnetic signal that is emitted from the tissue being imaged.

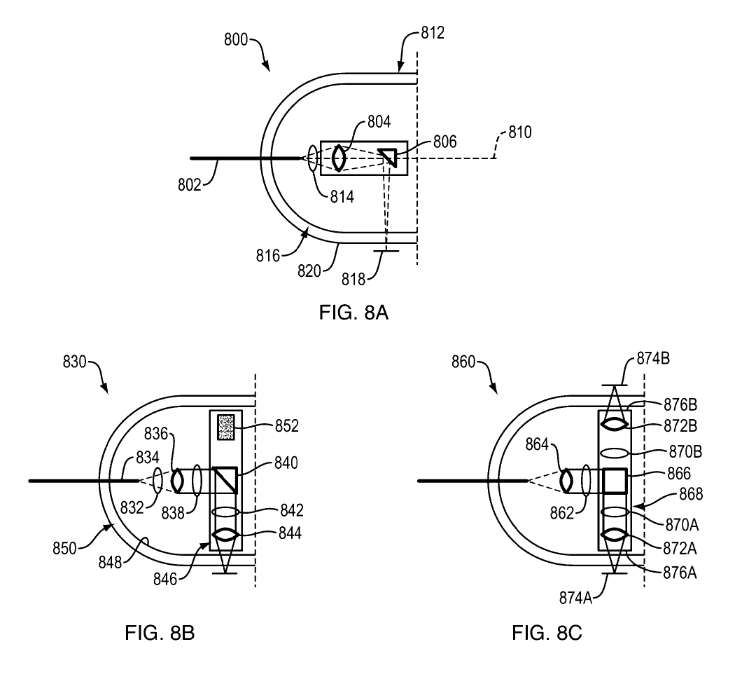

FIG. 8A though FIG. 8C are schematic diagrams showing exemplary embodiments of the focusing optics that transmits the optical beam from the optical fiber to the luminal wall adjacent to the enclosure. FIG. 8A shows one exemplary embodiment of a low NA focusing optics 800. The optical fiber 802, a focusing element 804, and a reflector 806 are aligned coaxially in the center axis 810 of the enclosure 812. The optical beam 814 emitted from the optical fiber 802 is focused by the focusing element 804 and reflected by the reflector 806 at a right angle through the wall 816 of the enclosure 812. In example embodiment, the focusing optics 800 is designed such that the focal plane 818 is less than 1 mm from the outer surface 820 of the enclosure 812. In this embodiment, the working distance of the system measured from the reflector is approximately equal the radius of the enclosure, which limits the maximum NA of the focusing element and thus the smallest possible focused beam spot size.

FIG. 8B shows one exemplary embodiment of a high NA focusing optics 830. The optical beam 832 emitted from the optical fiber 834 enters the focusing element 836 and exits as a collimated beam 838, which is directed at a right angle by a reflector 840. The reflected beam 842 is then focused by a second focusing element 844 that is held in the optics mount 846 at a short distance from the inner surface 848 of the enclosure 850. The optics mount 846 is held in a plane that is orthogonal to the path of the collimated optical beam 838. The short working distance of the second focusing element 844 enables it to be high NA and thereby produce a small focused beam spot size. The focusing element 844 is counterweighted on the opposing end of the optics mount 846 with a passive mass 852 in order to avoid vibration with rotary scanning.

FIG. 8C shows another embodiment of a high NA focusing optics 860. The collimated beam 862 emitted from focusing element 864 is split into two beams by a Wollaston prism or dichroic mirror or other beam splitting element 866 that is mounted in the center of an optics mount 868. Both output beams 870A and 870B from the beam splitting element 866 are separately focused by focusing elements 872A and 872B onto focal planes 874A and 874B on two ends of the optics mount, 876A and 876B, respectively.

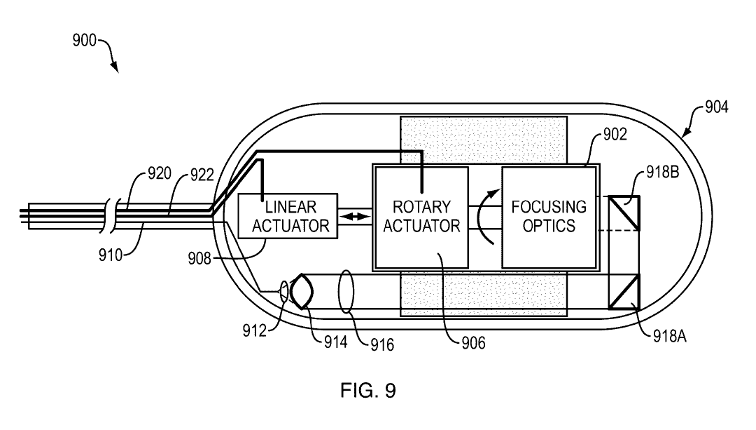

FIG. 9 shows an exemplary embodiment of the device 900. In the device 900, the focusing optics 902 is situated at the most distal end of the enclosure 904, such that the rotary actuator 906 and linear actuator 908 are more proximal. The optical fiber 910 projects an optical beam 912 into focusing element 914, which produces a collimated beam 916 that travels longitudinally over the length of the enclosure 904 and is multiply reflected by one or more reflectors 918A and 918B, such that the beam direction is reversed and the beam enters the focusing optics 902 from the distal end of the enclosure 904. This embodiment has the advantage that the control cables 920 and 922 for the linear actuator 908 and rotary actuator 906 do not obstruct the scanned circumference of the focusing optics 902 and are conveniently managed proximal to the enclosure 904.

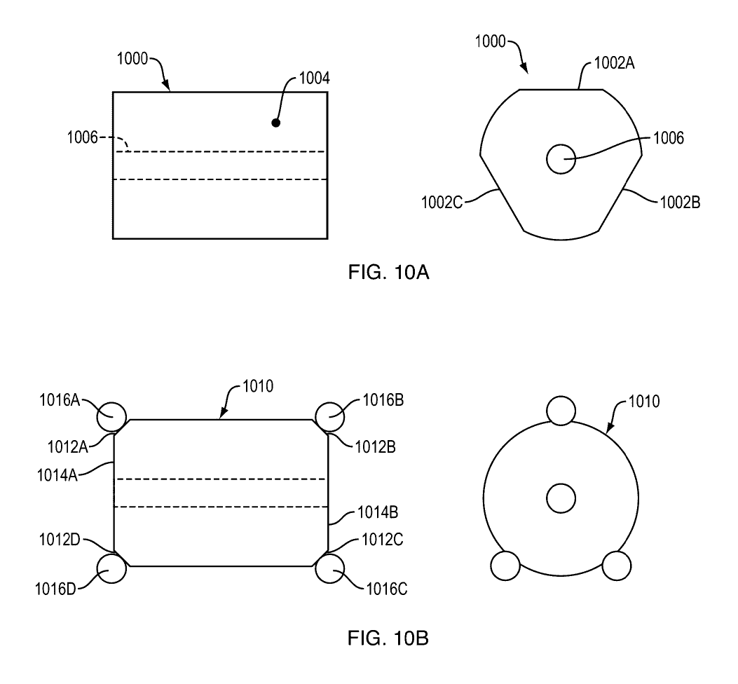

FIG. 10A and FIG. 10B are schematic diagrams showing two views of exemplary embodiments of a carriage that contains the rotary actuator (such as the carriages 314 in FIG. 3A, 366 in FIG. 3B, 468 in FIG. 4B, 516 in FIG. 5A 562 in FIG. 5B, 612 in FIG. 6A, and 714 in FIG. 7). The carriage has the requirement that it should translate across the length of the enclosure with minimal friction while holding the rotary actuator on the central longitudinal axis of the enclosure regardless of the enclosure orientation. In FIG. 10A, the carriage 1000 has multiple chamfers 1002A, 1002B, and 1002C along the longitudinal surface 1004, and a center through-hole 1006 for the rotary actuator. The carriage 1000 is manufactured from a low friction material such as Teflon or other material. The largest diameter of the carriage is determined to fit snugly within the enclosure inner diameter (not shown in FIG. 10A) such that the carriage 1000 is centered in the enclosure. The chamfers reduce the surface area of the carriage in contact with the enclosure to reduce frictional resistance during translation. In FIG. 10B, the carriage 1010 has multiple chamfers 1012A, through 1012D on both end surfaces 1014A and 1014B. Ball bearings 1016A through 1016D are mounted on the chamfers. Thus the carriage 1010 is in contact with the enclosure (not shown in FIG. 10B) on a number of points, which results in low friction translation along the inner surface of the enclosure.

The invention also contains methods and devices for correcting non-uniform or inaccurate beam scanning to obtain undistorted images. Both rotary and longitudinal scanning may be corrected. In one embodiment, the rotary actuator can have a sensor that indicates the rotary position of the beam position. In another embodiment, the rigid enclosure has multiple fiducial lines that are marked or embedded inside, or on the inner or outer surface of the enclosure that function as static landmarks for registration. The lines can be in several forms. As an example they can be a 1-dimensional pattern parallel to the longitudinal axis of the enclosure, or a two-dimensional pattern that covers the entire scanned area of the enclosure. The lines partially obstruct the beam when it is being scanned, producing a shadow on the images that allow the scans to be aligned in a known pattern during image reconstruction. The 1-dimensional pattern enables correction of consecutive rotary scans, while the 2-dimensional pattern enables correction of both rotary scanning and longitudinal scanning. Correction for the motion instabilities using the static landmarks requires extracting the position of the landmarks from the images and reconstructing the images with linear or non-linear resampling methods to align the motion-distorted patterns of the landmarks to the motion-free pattern. With both of the patterns it is possible to extract the precise rotary location of the static landmarks using a combination of interpolation, curve fitting and edge detection methods.

For the example of a 1-dimensional pattern with N horizontal lines there will be N anchor locations whose angular positions for each rotary image that can be extracted. Then according the Nyquist criteria, for a rotary frequency of f one can correct rotary instabilities upto a frequency f_{c} of:

$${f}_{c} = \frac{fN}{2} .$$

If the frequency content of the rotary instability is substantially contained within this frequency range then it should be possible to virtually eliminate all distortions caused by the rotational instability.

As an example, for the imaging modality of OCT, with a rotation frequency of 200 Hz and with 4 horizontal lines as static landmarks, one can correct for rotary instabilities that are occurring up to 400 Hz. Note that for the embodiments that employ slow rotary scan, the frequency of the rotary scan will be substantially low, hence one might require a large number of horizontal lines in case the system has a rotary instability at substantially larger frequencies than the rotary frequency.

An example of a 2-dimensional pattern can be N horizontal lines and another set of N_{c} lines that crosses the scanned area with a fixed angle with respect to the axial direction. For this case one can first extract the locations of the N horizontal lines from each rotary image and correct the images for rotary instabilities with the aforementioned method and constraints. This correction can be followed by the extraction of the location of the N_{c} cross-lines. In this case one would have N_{c} static landmark for each rotation, hence for a rotary frequency of f one can correct longitudinal scanning instabilities up to a frequency f_{c} of:

$${f}_{c} = \frac{{fN}_{c}}{2} .$$

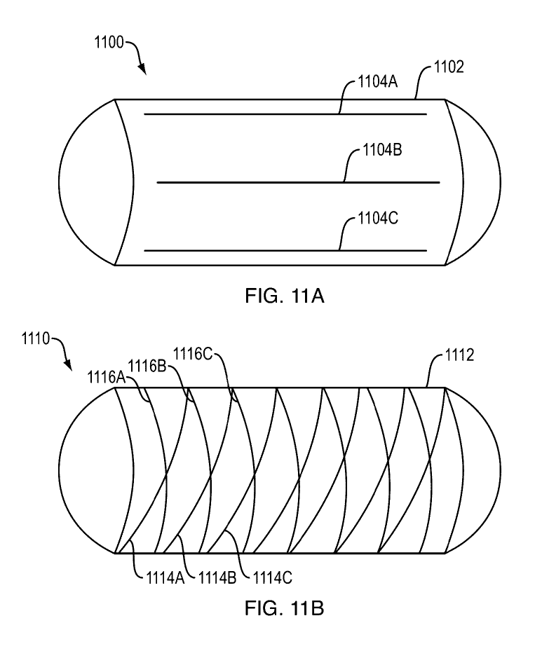

As an example, for the imaging modality of OCT, with a rotation frequency of 200 Hz and with 4 cross-lines as static landmarks, one can correct for longitudinal instabilities that are occurring up to 400 Hz. FIG. 11A and FIG. 11B are schematic diagrams showing exemplary embodiments of static landmarks for registration embedded inside, or on the outer or inner surface, or a combination therefore, of the enclosure wall that are used to correct inaccuracies in optical beam scanning. Referring to FIG. 11A, the enclosure 1100 includes a wall 1102 marked with an exemplary 1-dimensional repetitive pattern comprising fiducial marks 1104A, 1104B, 1104C, etc. The fiducial marks 1104A-C can be used to correct scan errors in 1 dimension that is either rotary or longitudinal scanning. Referring to FIG. 11B, the enclosure 1110 includes a wall 1112 marked with an exemplary 2-dimensional repetitive pattern comprising fiducial marks 1114A, B, C, etc., and 1116A, B, C, etc. Fiducial marks 1114 and 1116 can be used to correct scan errors in 2 dimensions, that is both rotary and linear scanning.

In yet another embodiment, with application to interferometry methods such as optical coherence tomography, the enclosure can have a microscopic variation in thickness, which enables determination of beam position. The thickness variation should not be large enough to cause excessive change in the focal position of the focusing optics. The thickness variation can be increased monotonically or periodically, and can occur in either or both the rotary or longitudinal direction of the enclosure. The enclosure of the device can have multiple layers with varying thickness such that both rotary and longitudinal position can be encoded and measured. In yet another embodiment, the reference arm of the interferometric system is varied in a known and highly accurate fashion, such that deviation in position of the interferometric signal is a measure of longitudinal position. Additionally, in the embodiment that contains a collimating beam prior to the optical focus, the variation in optical path length is a measure of longitudinal position.

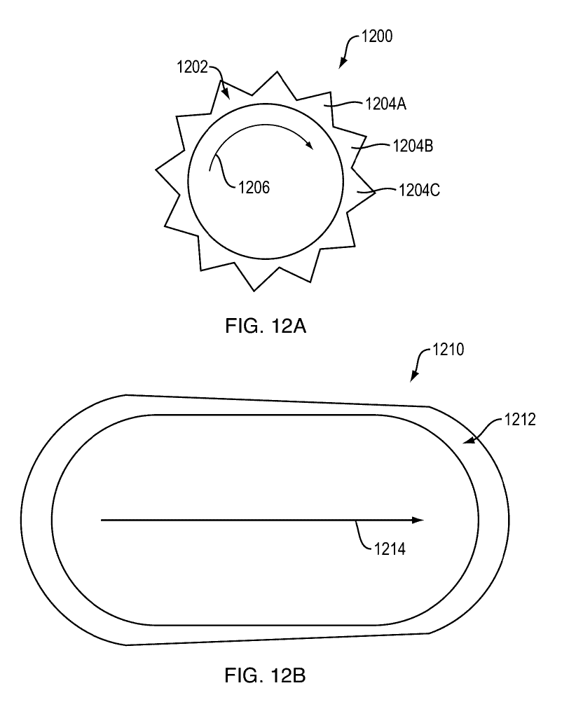

FIG. 12A and FIG. 12B are cross-sectional views of exemplary embodiments of enclosure cross sections illustrating variation in wall thickness of the enclosure that can be used to correct inaccuracies in optical beam scanning. The thickness variation is microscopic and does not significantly change the focal plane of the focusing optics. The thickness variation is detectable by optical interferometry methods, which can provide an indication of beam position along the dimension where the thickness is being varied. Referring to FIG. 12A, the enclosure 1200 includes a wall 1202. The wall 1202 includes regions of variable thickness 1204A, 1204B, 1204 C, etc. The thickness of the wall 1202 can vary in a periodic or aperiodic fashion along the rotary dimension denoted by arrow 1206. The thickness variation along the rotary direction 1206 can be used to correct scan inaccuracies by the rotary actuator. Referring to FIG. 12B, the enclosure 1210 includes a wall 1212. The thickness of the wall 1212 can vary in a periodic or aperiodic fashion in the longitudinal dimension denoted by arrow 1214. The measured thickness variation in the longitudinal direction can be used to correct scan inaccuracies by the linear actuator.

The device also can have mechanisms for stabilizing or detecting and correcting relative motion between the enclosure and the surrounding luminal tissue. In one embodiment, the enclosure has open ports on which a pneumatic vacuum is applied, resulting in the tissue surface being pulled towards the enclosure and the latter stabilized while achieving improved placement of tissue in the imaging focal plane. In another embodiment, the enclosure is encased in an inflatable balloon, which is inflated after the enclosure is introduced into the lumen, such that the enclosure is centered in the luminal organ. In yet another embodiment, an additional optical fiber or fiber bundle is placed adjacent to the enclosure in order to detect reflectance changes in the tissue surface, which is a measure of tissue motion relative to the enclosure.

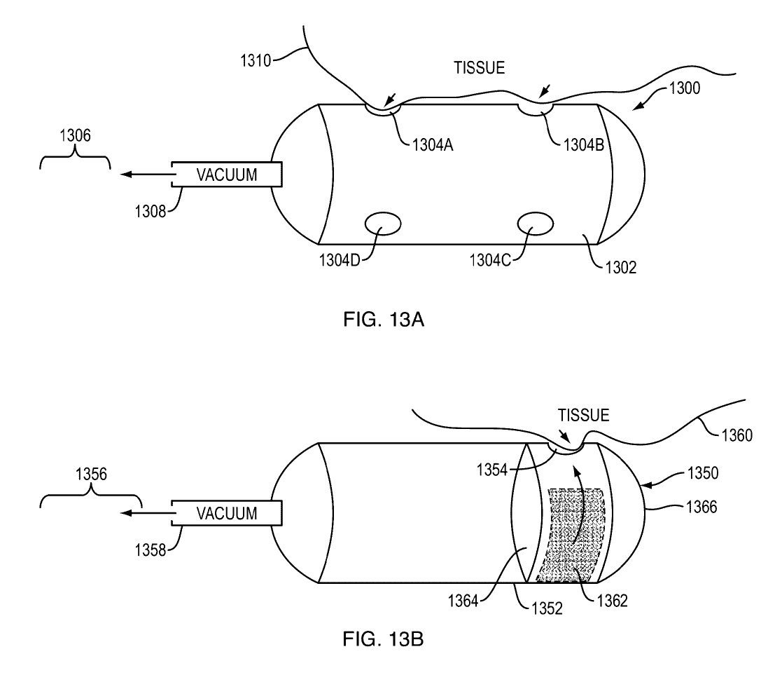

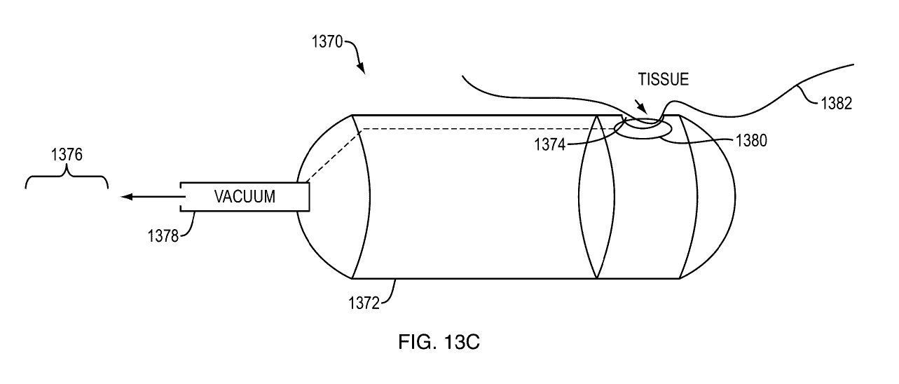

FIG. 13A is an illustration of an exemplary embodiment of vacuum stabilization functionality in the devices described herein. Referring to FIG. 13A, the enclosure 1300 includes a cylindrical surface 1302. Disposed on the cylindrical surface 1302 are open ports 1304A, 1304B, 1304C, etc. A vacuum exerted at the proximal end 1306 of the device via a tether 1308 (for example, catheter sheath) produces suction at the ports (vents) distributed around the cylindrical surface 1302 of the enclosure 1300. The suction pulls the surrounding tissue 1310 closer to the surface of the enclosure and into the focal plane of the focusing optics (not shown).

The invention also describes methods for integrated interventional procedures, such as tissue biopsy, mucosal resection, therapy, or other procedures. In one embodiment, the enclosure has one or more open ports to a separate chamber from which an applied vacuum pulls tissue into the ports, and a cutter blade is used to excise the tissue. The cutter can be actuated mechanically via a cable or torque cable from the proximal end of the device, or electromagnetically, pneumatically, or hydraulically at the distal end of the device. The cutter may be a planar, curved, rotary or other blade. The device may have multiple such chambers around the device, enabling multiple tissue biopsies or mucosal resections at different sites in a single imaging session. When a location of interest is identified during imaging, the device can be repositioned such that the port is in contact with the location of interest for excision to be performed. The location of interest can also be imaged prior to the excision. In another embodiment, a marking or therapy laser can be introduced using the optical fiber or an outer core of a dual clad optical fiber. The position of the marking or therapy is determined based on image data and analysis. In another embodiment, the enclosure has an electrocauterizing or other wire loop, also known as a snare, placed directly above the open port, such that tissue that is pulled into the port may be excised by tightening the wire loop. FIG. 13B is an illustration of an exemplary embodiment of an image-guided vacuum excision device for biopsy or mucosal resection or other interventional purpose.

Referring to FIG. 13B, the enclosure 1350 includes a wall 1352. Disposed in the wall 1352 is at least one open port 1354. A vacuum exerted at the proximal end 1356 of the device via a tether 1358 (for example, catheter sheath) produces suction at the at least one port (vent) 1354 of the enclosure 1350. A vacuum pulls an area of tissue into the port 1354. A rotary, curved or planar cutting blade 1362 is triggered to excise the suctioned-in tissue into an isolated chamber 1364. The cutting blade 1362 may be actuated by a cable, pressure or vacuum line, extending from the enclosure 1350 through the tether 1358 to the proximal end 1356. The cutting blade 1362 may also be actuated by a distal electromagnetic, piezoelectric or other mechanism which is proximally controlled (not shown). The size and depth of the port 1354 is chosen in order to excise a tissue sample of a desired size and to a desired depth. In one embodiment, the port 1354 can be located on the distal end 1366 of the enclosure 1350, while imaging is performed at a location more proximal to the longitudinal center of the enclosure 1350.

Imaging is first performed to identify a region of interest with potential pathology. In an embodiment with a single port, the port is located at a particular angular position on the cylindrical enclosure. The cylindrical enclosure is configured to be rotatable by the operator by torqueing the proximal end of the semi rigid tether. The rotation is used to position the port in line longitudinally with the region of interest containing possible pathology as determined by the imaging. Then the device is retracted a short distance in order to move the port longitudinally to coincide with the region of interest. The excision is performed by applying vacuum to pull the tissue region of interest into the port and actuating the cutting blade. The use of image guidance is expected to improve diagnostic sensitivity compared with procedures which use random tissue sampling. In a related embodiment, the port is located directly on a section of the transparent enclosure, such that imaging may be carried out with the port opening overlaid on the tissue area of interest. Tissue may then be excised via the open port, so that imaging may be exactly correlated with the excision site without a need to reposition the device or preemptively mark the site.

FIG. 13C is an illustration of another exemplary embodiment of an image-guided vacuum excision device for biopsy or mucosal resection or other interventional purpose. Referring to FIG. 13C, the enclosure 1370 includes a wall 1372. Disposed in the wall 1372 is at least one open port 1374. A vacuum exerted at the proximal end 1376 of the device via a tether 1378 (for example, catheter sheath) produces suction at the at least one port (vent) 1374 of the enclosure 1370. The enclosure 1370 further includes a wire loop 1380 that functions as an electrocauterizing or other type of snare. The tissue 1382 is then excised by the wire loop 1380.

Accordingly, in a first aspect of the invention, the invention is an apparatus for optical imaging of a luminal organ or surgical cavity. The apparatus comprises a proximal end, including at least one optical connection, and at least one of mechanical connection and an electrical connection, and a distal end that comprises a rigid enclosure. The rigid enclosure includes at least one transparent portion. The distal end includes at least one optical connection, and at least one of mechanical connection and an electrical connection. The apparatus further includes a flexible or semi-flexible tether. The tether includes at least one optical fiber that connects the distal end of the apparatus to the proximal end of the apparatus. The tether further connects at least one of a mechanical connection and an electrical connection of the proximal end to the at least one of mechanical connection and an electrical connection of the distal end. The rigid enclosure comprises at least one focusing optical element in optical communication with the at least one optical fiber. The focusing optical element is configured to direct and focus light from the optical fiber through the transparent portion of the rigid enclosure. The apparatus further includes a scanning mechanism. The scanning mechanism including a rotary actuator configured to perform beam scanning in a rotary direction. The scanning mechanism is further configured to perform beam scanning in a longitudinal direction, wherein the rotary direction and the longitudinal direction are non-parallel.

In the second aspect of the present invention, the apparatus of the present invention is as described above with respect to the first aspect of the invention and can further include, as a part of its scanning mechanism, a linear actuator configured to perform beam scanning in a longitudinal direction. In any embodiment of the apparatus of the present invention that includes a linear actuator, the linear actuator can be disposed at the proximal end of the apparatus, and be configured to transfer linear motion to the torque cable, thereby producing a longitudinal beam scanning. In any embodiment of the present invention that includes a linear actuator, the linear actuator can be a pneumatic or a hydraulic actuator.

In an example embodiment of the second aspect of the invention, the present invention is an apparatus as described above with respect to the second aspects of the invention and its example embodiments, wherein the rotary actuator and the linear actuator are both disposed within the rigid enclosure. In an example embodiment of the second aspect, the rotary actuator is configured to produce a rapid rotary beam scan, and the linear actuator is configured to produce a slow longitudinal beam scan. As stated above, in any embodiment of the present invention that includes a linear actuator, the linear actuator can be a pneumatic or a hydraulic actuator.

In a third aspect, the present invention is an apparatus as described above with respect to the first aspect and any of its example embodiments, wherein the rotary actuator is disposed within the rigid enclosure. In example embodiments of the third aspect, the rotary actuator is configured to produce a rapid rotary beam scan, and is in mechanical communication with a mechanical transducer. The mechanical transducer being configured to transduce rotary motion to longitudinal motion, and to produce a slow longitudinal beam scan.

In a fourth aspect, the present invention is an apparatus as described above with respect to the first aspect, wherein the rotary actuator is disposed at the proximal end of the apparatus. In an example embodiment of the fourth aspect, the rotary actuator is configured to actuate a torque cable disposed within the tether. The torque cable is configured to transfer rotary motion from the proximal end of the apparatus to the distal end of the apparatus. In an example embodiment of the fourth aspect, the torque cable is in mechanical communication with a rotary frequency changing mechanism, the rotary frequency changing mechanism being in mechanical communication with the at least one focusing optical element, the rotary frequency changing mechanism being configured to produce a rapid rotary beam scanning. In an example embodiment of the fourth aspect, the apparatus further includes a mechanical transduction mechanism, the rotary actuator being configured to produce a slow longitudinal scan.

In a fifth aspect, the invention is an apparatus as described with respect to the first, the second, the third, and the fourth aspects and any of their example embodiments, and further wherein at least one focusing optical element is a low numerical aperture element or a high numerical aperture element. In an example embodiment of the fifth aspect, the rigid enclosure has a central longitudinal axis, and wherein the at least one focusing optical element is a low numerical aperture element having an optical axis aligned on the central longitudinal axis. In another example embodiment of the fifth aspect, the rigid enclosure has a characteristic radius, and at least one focusing optical element is a high numerical aperture element having a focal distance smaller than the characteristic radius of the rigid enclosure. Furthermore, at least one focusing optical element has an optical axis aligned perpendicular to the central axis of the rigid enclosure.

In a sixth aspect, the invention is an apparatus as described with respect to the first, the second, the third, the fourth, and the fifth aspects and any of their example embodiments, and further wherein the rigid enclosure includes at least one beam-splitting element configured to separate an input beam into multiple output beams. In an example embodiment of the fifth aspect, the beam-splitting element divides the input beam into multiple beams of different phase or different polarization. In any example embodiment of the sixth aspect, the rigid enclosure can include optical elements configured to produce two or more beams having an optical path difference.

In a seventh aspect, the invention is an apparatus as described with respect to the first, the second, the third, the fourth, the fifth, and the sixth aspects and any of their example embodiments, and further the rigid enclosure can include at least one static landmark configured to detect and correct non-uniform or inaccurate beam scanning.

In an eighth aspect, the invention is an apparatus as described with respect to the first, the second, the third, the fourth, the fifth, the sixth, and the seventh aspects and any of their example embodiments, and further wherein the apparatus includes a tissue biopsy extractor or an endoscopic mucosal resection tool.

In an ninth aspect, the invention is an apparatus as described with respect to the first, the second, the third, the fourth, the fifth, the sixth, the seventh, and the eighth aspects and any of their example embodiments, and further wherein the apparatus includes a laser marking mechanism configured to mark tissue.

In a tenth aspect, the invention is an apparatus as described with respect to the first, the second, the third, the fourth, the fifth, the sixth, the seventh, the eighth, and the ninth aspects and any of their example embodiments, and further wherein the rigid enclosure includes at least one region of variable thickness configured to identify a scanning beam position.

In an eleventh aspect, the invention is an apparatus as described with respect to the first, the second, the third, the fourth, the fifth, the sixth, the seventh, the eighth, the ninth, and the tenth aspects and any of their example embodiments, and further wherein the apparatus includes at least one sensor configured to measure a scanning beam position.

In an twelfth aspect, the invention is an apparatus as described with respect to the first, the second, the third, the fourth, the fifth, the sixth, the seventh, the eighth, the ninth, the tenth, and the eleventh aspects and any of their example embodiments, and further wherein the apparatus includes a stabilization mechanism for stabilizing relative motion between the rigid enclosure and a surrounding tissue. In an example embodiment of the twelfth aspect, the stabilization mechanism includes an inflatable balloon, the rigid enclosure being contained within said inflatable balloon, the inflatable balloon being configured to inflate and stabilize the position of the rigid enclosure within the luminal organ or the surgical cavity. In another example embodiment of the twelfth aspect, the stabilization mechanism includes a pneumatic vacuum generator, the rigid enclosure further including at least one port configured to apply pneumatic vacuum to a surrounding tissue.

In a thirteenth aspect, the invention is an apparatus as described with respect to the first, the second, the third, the fourth, the fifth, the sixth, the seventh, the eighth, the ninth, the tenth, the eleventh, and the twelfth aspects and any of their example embodiments, and further wherein the apparatus includes a tissue reflectance detection module disposed adjacent to the rigid enclosure, the tissue reflectance detection module being configured to detect relative motion between the rigid enclosure and a surrounding tissue.

In a fourteenth aspect, the present invention is an apparatus for optical imaging of a luminal organ or surgical cavity. The apparatus comprises a proximal end that includes at least one optical connection, and at least one of mechanical connection and an electrical connection. The apparatus further includes a distal end that comprises a rigid enclosure. The rigid enclosure includes at least one transparent portion. The distal end includes at least one optical connection, and at least one of mechanical connection and an electrical connection. The apparatus further includes a flexible or semi-flexible tether. The tether includes at least one optical fiber that connects the distal end of the apparatus to the proximal end of the apparatus. The tether further connects at least one of a mechanical connection and an electrical connection of the proximal end to the at least one of mechanical connection and an electrical connection of the distal end. The rigid enclosure further comprises at least one focusing optical element in optical communication with the at least one optical fiber. The focusing optical element is configured to direct and focus light from the optical fiber through the transparent portion of the rigid enclosure. The apparatus further includes a scanning mechanism, the scanning mechanism including a rotary actuator, the rotary actuator being configured to perform beam scanning in a rotary direction. In an example embodiment of the fourteenth aspect of the invention, the rigid enclosure includes at least one static landmark configured to detect and correct non-uniform or inaccurate beam scanning. In any of the example embodiments of the fourteenth aspect, the optical fiber can remain stationary in the rotary direction with respect to the rigid enclosure during beam scanning.

In another example embodiment of the 14^{th} aspect of the present invention, the rotary actuator is disposed at the proximal end of the apparatus, the rotary actuator being configured to actuate a torque cable disposed within the tether. The torque cable is configured to transfer rotary motion from the proximal end of the apparatus to the distal end of the apparatus. The torque cable is in mechanical communication with a rotary frequency changing mechanism, the rotary frequency changing mechanism being in mechanical communication with the at least one focusing optical element, the rotary frequency changing mechanism is configured to produce a rapid rotary beam scanning.

In any of the example embodiment of any aspect of the present invention, the rotary actuator can be a pneumatic or a hydraulic actuator.

In a 15^{th} aspect, the invention is an apparatus as described with respect to the 14^{th} aspect and any of its example embodiments, and further wherein the scanning mechanism is further configured to perform beam scanning in a longitudinal direction, wherein the rotary direction and the longitudinal direction are non-parallel. In an example embodiment of the 15^{th} aspect, the scanning mechanism includes a movable carriage disposed within the rigid enclosure, the movable carriage being in mechanical communication with the torque cable, the at least one focusing optical element being disposed on the movable carriage, the movable carriage being configured to move in a longitudinal direction within the rigid enclosure.

In a 16^{th} aspect, the invention is an apparatus as described with respect to the 14^{th} and 15^{th} aspects and any of their example embodiments, and further wherein the focusing optical element is a low numerical aperture element or a high numerical aperture element. In an example embodiment of the 16^{th} aspect, the rigid enclosure has a central longitudinal axis, and at least one focusing optical element is a low numerical aperture element having an optical axis aligned on the central longitudinal axis. In another example embodiment of the 16^{th} aspect, the rigid enclosure has a characteristic radius, and at least one focusing optical element is a high numerical aperture element having a focal distance smaller than the characteristic radius of the rigid enclosure, the at least one focusing optical element having an optical axis aligned perpendicular to the central axis of the rigid enclosure.

In a 17^{th} aspect, the invention is an apparatus as described with respect to the 14^{th}, 15^{th}, and 16^{th} aspects and any of their example embodiments, and further wherein the rigid enclosure includes at least one beam-splitting element configured to separate an input beam into multiple output beams. In an example embodiment of the 17^{th} aspect, the beam-splitting element divides the input beam into multiple beams of different phase or polarization. In another example embodiment of the 17^{th} aspect, the rigid enclosure includes optical elements configured to produce two or more beams having an optical path difference.

In a 18^{th} aspect, the invention is an apparatus as described with respect to the 14^{th}, 15^{th}, 16^{th}, and the 17^{th} aspects and any of their example embodiments, and further wherein the apparatus comprises a tissue biopsy extractor or an endoscopic mucosal resection tool.

In a 19^{th} aspect, the invention is an apparatus as described with respect to the 14^{th}, 15^{th}, 16^{th}, 17^{th}, and the 18^{th} aspects and any of their example embodiments, and further wherein the apparatus comprises a laser marking mechanism configured to mark tissue.

In a 20^{th} aspect, the invention is an apparatus as described with respect to the 14^{th}, 15^{th}, 16^{th}, 17^{th}, 18^{th}, and the 19^{th} aspects and any of their example embodiments, and further wherein the rigid enclosure includes at least one region of variable thickness configured to identify a scanning beam position.

In a 21^{st} aspect, the invention is an apparatus as described with respect to the 14^{th}, 15^{th}, 16^{th}, 17^{th}, 18^{th}, 19^{th}, and the 20^{th} aspects and any of their example embodiments, and further wherein the apparatus includes at least one sensor configured to measure a scanning beam position.

In a 22^{nd} aspect, the invention is an apparatus as described with respect to the 14^{th}, 15^{th}, 16^{th}, 17^{th}, 18^{th}, 19^{th}, 20^{th}, and the 21^{st} aspects and any of their example embodiments, and further wherein the apparatus includes a stabilization mechanism for stabilizing relative motion between the rigid enclosure and a surrounding tissue.

In a 23^{rd} aspect, the invention is an apparatus as described with respect to the 14^{th}, 15^{th}, 16^{th}, 17^{th}, 18^{th}, 19^{th}, 20^{th}, 21^{st}, and the 22^{nd} aspects and any of their example embodiments, and further wherein the apparatus includes a tissue reflectance detection module disposed adjacent to the rigid enclosure, the tissue reflectance detection module being configured to detect relative motion between the rigid enclosure and a surrounding tissue.

In further aspects, the present invention includes methods of using the devices and apparatuses described above with respect to any of the 1^{st} through 23^{rd} aspects and any of their example embodiments.

For example, in a 24^{th} aspect, the present invention is a method of optical imaging of a luminal organ or a surgical cavity. The method comprises providing any of the apparatuses described above with respect to any of the 1^{st} through 23^{rd} aspects and any of their example embodiments, causing the apparatus to scan the luminal organ or surgical cavity, and acquiring the optical image of the luminal organ or surgical cavity.

In a 25^{th} aspect, the present invention is a method for correcting an optical image of a luminal organ or a surgical cavity. The method comprises providing any of the 1^{st} through 23^{rd} aspects and any of their example embodiments, causing the apparatus to scan the luminal organ or surgical cavity and to acquire the optical image of the luminal organ or surgical cavity, detecting a scanning beam position, measuring inaccuracies in the scanning beam position, and, based on the measured inaccuracies in the scanning beam position, controlling the at least one scanning mechanism to correct the optical image.

In example embodiments of either the 24^{th} or the 25^{th} aspects of the present invention, the method can further include one or more of the following operations:
causing the apparatus to scan the luminal organ or surgical cavity includes linearly translating the rigid enclosure;
registering the acquired optical image relative to the at least one static landmark;
identifying the scanning beam position relative to the at least one region of variable thickness;
causing at least one sensor to measure the scanning beam position within the luminal organ or surgical cavity;
marking a position of interest within the luminal organ or the surgical cavity with a laser beam using the laser marking mechanism;
stabilizing relative motion between the rigid enclosure and the surrounding tissue using the stabilization mechanism; and
detecting a change in reflectance of the surrounding tissue using the tissue reflectance detection module, thereby detecting relative motion between the rigid enclosure and the surrounding tissue;

### EXEMPLIFICATION

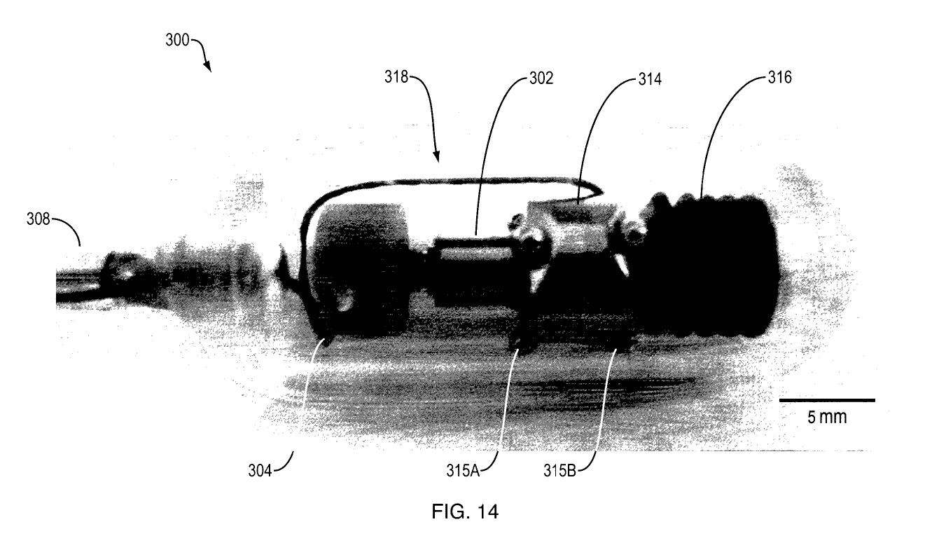

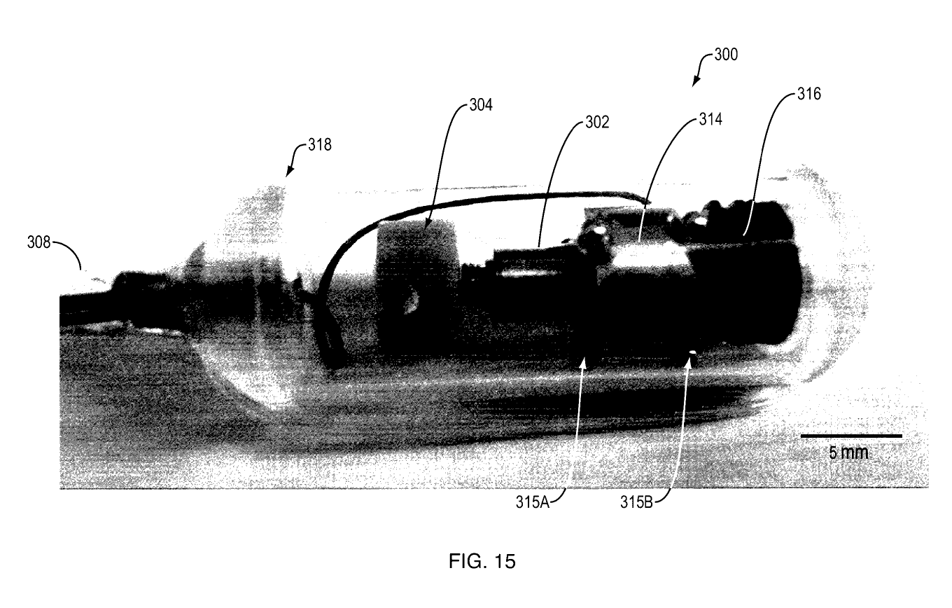

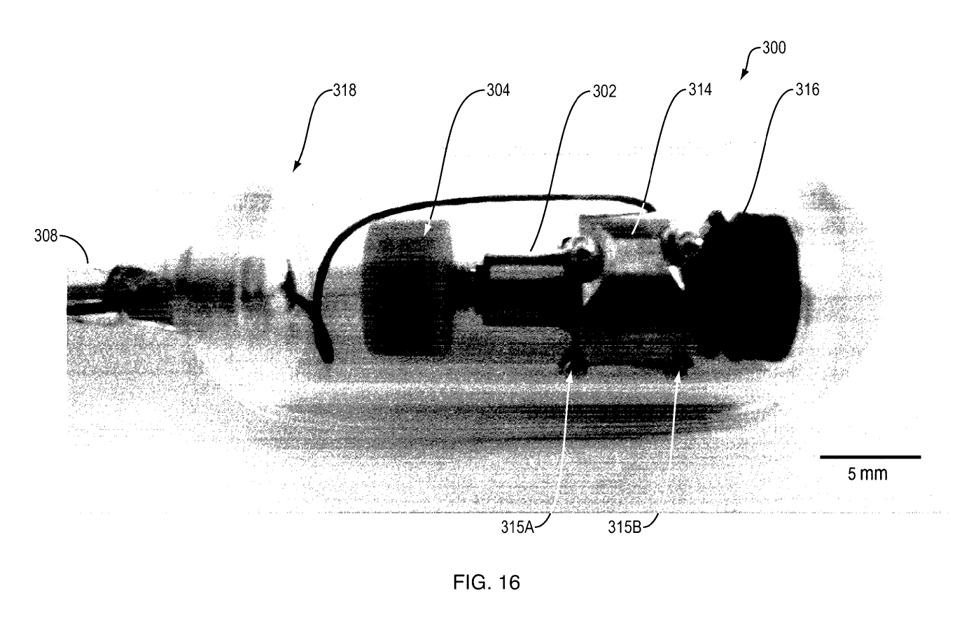

FIG. 14, FIG. 15, and FIG. 16 are the photographs depicting an embodiment of the device of the present invention. The scale bar is 5 mm. A schematic of this particular embodiment 300 as presented in FIG. 3A. In FIG. 14, a distal rotary actuator 302 that is an electromagnetic motor rotates the focusing optics 304 and is mounted in a carriage 314 that slides on ball bearings 315A, 315B, etc. The carriage 314 is also mounted longitudinally to a pneumatic actuator (linear) 316 that includes bellows. An optical fiber and collimating lens (not shown in FIGS. 14 through 16) are mounted in the proximal cap of the enclosure and is aligned to the focusing optics, which in this embodiment is the high NA configuration. The control cable for the rotary actuator and the inflation tube for the pneumatic actuator (not shown in FIGS. 14-16) return to the proximal end via the tether 308. In this embodiment, the inflation tube is outside the enclosure; however, this may also be placed inside the enclosure as described previously.

In FIG. 15, the pneumatic actuator is contracted by exsufflating the bellows, and the carriage is thus translated longitudinally towards the distal end of the enclosure.

In FIG. 16, the rotary actuator is rotating the focusing optics.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. An apparatus for optical imaging of a luminal organ or surgical cavity, the apparatus comprising:
a proximal end including at least one optical connection, and at least one of mechanical connection and an electrical connection;
a distal end that comprises a rigid enclosure, the rigid enclosure including at least one transparent portion, the distal end including at least one optical connection, and at least one of mechanical connection and an electrical connection;
a flexible or semi-flexible tether, the tether including at least one optical fiber that connects the distal end of the apparatus to the proximal end of the apparatus, the tether further connecting at least one of a mechanical connection and an electrical connection of the proximal end to the at least one of mechanical connection and an electrical connection of the distal end;
the rigid enclosure further comprising at least one focusing optical element in optical communication with the at least one optical fiber, the at least one focusing optical element configured to direct and focus light from the optical fiber through the transparent portion of the rigid enclosure; and
a scanning mechanism, the scanning mechanism including a rotary actuator configured to perform beam scanning in a rotary direction, the scanning mechanism further being configured to perform beam scanning in a longitudinal direction, wherein the rotary direction and the longitudinal direction are non-parallel.

2. The apparatus of claim 1, wherein the scanning mechanism further includes a linear actuator configured to perform beam scanning in a longitudinal direction.

3. The apparatus of claim 2, wherein the rotary actuator and the linear actuator are disposed within the rigid enclosure, and further wherein:
the rotary actuator is configured to produce a rapid rotary beam scan; and
the linear actuator is configured to produce a slow longitudinal beam scan.

4. The apparatus of claim 3, wherein the linear actuator is a pneumatic or a hydraulic actuator.

5. (canceled)

6. The apparatus of claim 1, wherein the rotary actuator is disposed within the rigid enclosure, and further wherein:
the rotary actuator is configured to produce a rapid rotary beam scan; and
the rotary actuator is in mechanical communication with a mechanical transducer, the mechanical transducer being configured to transduce rotary motion to longitudinal motion, and to produce a slow longitudinal beam scan.

7. The apparatus of claim 1, wherein:
the rotary actuator is disposed at the proximal end of the apparatus, the rotary actuator being configured to actuate a torque cable disposed within the tether, and further wherein:
the torque cable is configured to transfer rotary motion from the proximal end of the apparatus to the distal end of the apparatus,
the torque cable being in mechanical communication with a rotary frequency changing mechanism, the rotary frequency changing mechanism being in mechanical communication with the at least one focusing optical element, the rotary frequency changing mechanism being configured to produce a rapid rotary beam scanning.

8. The apparatus of claim 7, further including a mechanical transduction mechanism, the rotary actuator being configured to produce a slow longitudinal scan.

9. The apparatus of claim 1, wherein the at least one focusing optical element is a low numerical aperture element or a high numerical aperture element.

10. The apparatus of claim 9, wherein the rigid enclosure has a central longitudinal axis, and wherein the at least one focusing optical element is a low numerical aperture element having an optical axis aligned on the central longitudinal axis.

11. The apparatus of claim 9, wherein the rigid enclosure has a characteristic radius, and wherein the at least one focusing optical element is a high numerical aperture element having a focal distance smaller than the characteristic radius of the rigid enclosure, the at least one focusing optical element having an optical axis perpendicular to the central axis of the rigid enclosure.

12. The apparatus of claim 1, wherein the rigid enclosure includes at least one beam-splitting element configured to separate an input beam into multiple output beams.

13. The apparatus of claim 12, wherein the beam-splitting element divides the input beam into multiple beams of different phase or different polarization.

14. The apparatus of claim 12, wherein the rigid enclosure includes optical elements configured to produce two or more beams having an optical path difference.

15. The apparatus of claim 1, wherein the rigid enclosure includes at least one static landmark configured to detect and correct non-uniform or inaccurate beam scanning.

16. The apparatus of claim 1, further comprising a tissue biopsy extractor or an endoscopic mucosal resection tool.

17. The apparatus of claim 1, further comprising a laser marking mechanism configured to mark tissue.

18. The apparatus of claim 1, wherein the rigid enclosure includes at least one region of variable thickness configured to identify a scanning beam position.

19. The apparatus of claim 1, further including at least one sensor configured to measure a scanning beam position.

20. The apparatus of claim 1, further including a stabilization mechanism for stabilizing relative motion between the rigid enclosure and a surrounding tissue.

21. The apparatus of claim 20, wherein the stabilization mechanism includes a pneumatic vacuum generator, the rigid enclosure further including at least one port configured to apply pneumatic vacuum to a surrounding tissue.

22. (canceled)

23. The apparatus of claim 1, further including a tissue reflectance detection module disposed adjacent to the rigid enclosure, the tissue reflectance detection module being configured to detect relative motion between the rigid enclosure and a surrounding tissue.

24.-40. (canceled)

41. A method of optical imaging of a luminal organ or a surgical cavity, comprising:
providing an apparatus of claim 1;
causing the apparatus to scan the luminal organ or surgical cavity; and
acquiring the optical image of the luminal organ or surgical cavity.

42.-48. (canceled)

49. A method for correcting an optical image of a luminal organ or a surgical cavity, comprising:
providing an apparatus of claim 1;
causing the apparatus to scan the luminal organ or surgical cavity, and to acquire the optical image of the luminal organ or surgical cavity;
detecting a scanning beam position;
measuring inaccuracies in the scanning beam position; and
based on the measured inaccuracies in the scanning beam position, controlling the at least one scanning mechanism to correct the optical image.

50.-52. (canceled)
